(19) United States
(12) Patent Application Publication
(10) Pub. No.: US 2024/0426840 A1
(43) Pub. Date: Dec. 26, 2024

(54) COMPOSITIONS AND METHODS FOR DETECTING AND TREATMENT OF MEMBRANOPROLIFERATIVE GLOMERULONEPHRITIS

(71) Applicant: The Texas A&M University System, College Station, TX (US)

(72) Inventors: Jessica A. Hokamp, Bryan, TX (US); Mary B. Nabity, College Station, TX (US)

(21) Appl. No.: 18/747,285

(22) Filed: Jun. 18, 2024

Related U.S. Application Data
(60) Provisional application No. 63/509,189, filed on Jun. 20, 2023.

(51) Int. Cl. G01N 33/68; A61K 31/192; A61K 31/5377; A61K 31/573; A61K 38/13; A61K 45/06; G01N 27/447; G01N 33/493

(52) U.S. Cl. CPC G01N 33/6893; A61K 31/192; A61K 31/5377; A61K 31/573; A61K 38/13; A61K 45/06; G01N 27/44721; G01N 33/493; G01N 2800/24

(57) ABSTRACT

Disclosed are methods and compositions for detecting glomerular disease in dogs and effectively treating dogs with glomerular diseases such as membranoproliferative immune complex-mediated glomerulonephritis (MPGN). Methods and compositions for detecting glomerular or tubular damage in cats and effectively treating cats with glomerular or tubular damage are also provided.

### CROSS-REFERENCE TO RELATED APPLICATIONS

This application claims the benefit of U.S. provisional application No. 63/509,189, filed Jun. 20, 2023, herein incorporated by reference in its entirety.

### STATEMENT REGARDING FEDERALLY SPONSORED RESEARCH OR DEVELOPMENT

This invention was made with government support under grant NIH T35 OD010991 awarded by the National Institutes of Health. The government has certain rights in the invention.

### FIELD OF THE INVENTION

The present invention relates generally to the field of veterinarian sciences. More specifically, the invention relates to methods and compositions for detecting membranoproliferative glomerulonephritis and treating the same.

### BACKGROUND OF THE INVENTION

Chronic kidney disease (CKD) is a significant cause of morbidity and mortality in all breeds of dogs and cats, and it is commonly caused by underlying glomerular diseases. Common glomerular diseases in dogs with naturally occurring chronic kidney disease (CKD) include amyloidosis (AMYL), glomerulosclerosis (GS), and immune complex-mediated glomerulonephritis (ICGN). ICGN can be further defined as: membranous glomerulonephritis (MGN), membranoproliferative glomerulonephritis (MPGN), and mesangioproliferative glomerulonephritis (MesGN). Each of these glomerular diseases has different treatment and prognostic considerations.

Proper treatment relies on an accurate diagnosis of the type of glomerular disease. However, there are currently no non-invasive or minimally-invasive tests to reliably diagnose the category, including subcategory, of glomerular disease. Renal biopsy and comprehensive pathologic examination are currently required for diagnosing specific glomerular diagnostic categories and guiding proper treatments. However, such procedures are not undertaken for the majority of dogs and cats because the patient is either deemed unsuitable for anesthesia and/or the cost of the renal biopsy is prohibitive. Despite the current treatment recommendations, there is a lack of evidence-based therapies that take into account the specific category of glomerular disease. Furthermore, without the aid of a renal biopsy, current treatment recommendations may rely on incorrect assumptions and lead to unintentional negative outcomes. Thus, there remains a need to provide methods and compositions for accurate detection paired with targeted treatment of glomerular disease, especially in companion animals such as dogs and cats.

### SUMMARY OF THE INVENTION

One aspect of the present invention provides a method of detecting membranoproliferative immune complex-mediated glomerulonephritis (MPGN) in a dog or cat comprising detecting in a sample from said dog or cat the presence of at least two high molecular weight proteins in the sample. In one embodiment, the proteins have a molecular weight greater than or equal to about 200 kDa; at least one protein in the sample has a molecular weight greater than or equal to about 300 kDa; or an RSA200 value greater than or equal to about 0.7% in the sample, wherein said detecting is indicative of MPGN in the dog or cat. In certain further embodiments, a high molecular weight protein detected in accordance with the invention to indicate the presence of MPGN, has a molecular weight of at least about 125 kDa, 150 kDa, 175 kDa, 200 kDa, 225 kDa, 250 kDa, 275 kDa, 300 kDa, 325 kDa, 350 kDa or 375 kDa.

In one embodiment, detecting a high molecular weight protein in accordance with the invention comprises performing electrophoresis on the sample. In particular embodiments, performing electrophoresis comprises sodium dodecyl-sulfate polyacrylamide gel electrophoresis. In further embodiments, detecting comprises staining the polyacrylamide gel with a gel staining reagent. In specific embodiments, sodium dodecyl-sulfate polyacrylamide gel electrophoresis comprises electrophoresis on a 4-12% Bis-Tris polyacrylamide gel; or performing electrophoresis at about 200 V. In some embodiments, the concentration of the protein in the sample is at least 3 ng/μL. In particular embodiments, wherein when relative surface area of the protein is quantified, the RSA value is greater than or equal to 0.01%, 0.02%, 0.025%, 0.05%, 0.075%, 0.1%, 0.2%, 0.3%, 0.4%, or 0.5%. In some embodiments, the method comprises detecting the presence of at least two proteins in the sample having a molecular weight greater than or equal to 200 kDa; and wherein the at least two proteins are detectable by SDS-PAGE using a conventional gel staining reagent. In other embodiments, the method comprises detecting the presence of at least one protein in the sample having a molecular weight greater than or equal to 300 kDa; and wherein the at least one protein is detectable by SDS-PAGE using a conventional gel staining reagent. In certain embodiments, such methods comprise detecting the presence of at least three, four, five, six, or seven proteins in the sample having a molecular weight greater than or equal to 200 kDa; detecting the presence of at least two proteins in the sample having a molecular weight greater than or equal to 300 kDa; or detecting an RSA200 value greater than or equal to 0.8%, 0.9%, or 1.0%. In some embodiments, the sample is a urine sample. In other embodiments, the method further comprises diluting the sample to normalize the sample to urine specific gravity.

In another aspect, the invention provides a method of treating a dog or cat with membranoproliferative immune complex-mediated glomerulonephritis (MPGN) comprising obtaining a sample from said dog or cat and detecting the presence of at least two proteins in the sample having a molecular weight greater than or equal to 200 kDa, at least one protein in the sample having a molecular weight greater than or equal to 300 kDa, or an RSA200 value greater than or equal to 0.7% in the sample; and treating the dog or cat based on the said detection. In one embodiment, detecting comprises performing electrophoresis on the sample. In particular embodiments, performing electrophoresis comprises sodium dodecyl-sulfate polyacrylamide gel electrophoresis. In further embodiments, detecting comprises staining the polyacrylamide gel with a gel staining reagent. In some embodiments, treating the dog or cat comprises no treatment; or does not comprise immunosuppressive therapy. In other embodiments, treating the dog or cat comprises immunosuppressive therapy; or angiotensin-converting enzyme (ACE) inhibitors (e.g., enalapril, benazepril, captopril, ramipril), angiotensin receptor blockers (ARBs; e.g., telmisartan, losartan), managing of blood pressure (e.g., beta-blockers, calcium channel inhibitors), anticoagulants (e.g., aspirin, clopidogrel, rivaroxaban), omega-3 fatty acid supplementation, or use of specially formulated diets for renal disease patients. In specific embodiments, the immunosuppressive therapy comprises mycophenolate, cyclophosphamide, azathioprine, chlorambucil, or cyclosporine. In some embodiments, the sample is a urine sample. In other embodiments, the method further comprises diluting the sample to normalize the sample to urine specific gravity. In specific embodiments, the concentration of the protein in the sample is at least 3 ng/μL. In particular embodiments, wherein when relative surface area of the protein is quantified, the RSA value is greater than or equal to 0.01, 0.02%, 0.025%, 0.05%, 0.075%, 0.1%, 0.2%, 0.3%, 0.4%, or 0.5%.

In yet another aspect, the invention provides a method of treating a dog or cat with membranoproliferative immune complex-mediated glomerulonephritis (MPGN) comprising obtaining a sample from the dog or cat that comprises at least a first protein associated with MPGN; detecting the presence of the at least first protein associated with MPGN in the sample, wherein said protein comprises a molecular weight greater than or equal to 200 kDa; and treating the dog or cat based on the presence of said protein in the sample. In certain embodiments, the detecting comprises separating the at least first protein associated with MPGN in the sample based on molecular weight.

In yet a further aspect, the invention provides A method of detecting glomerular or tubular damage in a cat comprising detecting in a sample from said cat the presence of: at least two proteins in the sample having a molecular weight less than or equal to 40 kDa; at least four proteins in the sample having a molecular weight between about 40 kDa and 70 kDa; at least two proteins in the sample having a molecular weight greater than or equal to 70 kDa in the sample, wherein said detecting is indicative of glomerular or tubular damage in the cat. In some embodiments, detecting comprises performing electrophoresis on the sample. In other embodiments, performing electrophoresis comprises sodium dodecyl-sulfate polyacrylamide gel electrophoresis. In still further embodiments, the method comprises detecting the presence of: at least three proteins in the sample having a molecular weight less than or equal to 40 kDa; at least five proteins in the sample having a molecular weight between about 40 kDa and 70 kDa; or at least three proteins in the sample having a molecular weight greater than or equal to 70 kDa; and wherein the proteins are detectable by SDS-PAGE using a conventional gel staining reagent. In some embodiments, the sample is a urine sample.

In another aspect, the invention provides a method of treating a cat with glomerular or tubular damage comprising: obtaining a sample from said cat; detecting in the sample the presence of: at least two proteins in the sample having a molecular weight less than or equal to 40 kDa; at least four proteins in the sample having a molecular weight between about 40 kDa and 70 kDa; or at least two proteins in the sample having a molecular weight greater than or equal to 70 kDa; and treating the cat based on the presence thereof. In some embodiments, treating the cat: comprises no treatment; comprises immunosuppressive therapy; or does not comprise immunosuppressive therapy. In particular embodiments, treating the cat comprises administering immunosuppressive therapy, angiotensin-converting enzyme (ACE) inhibitors (e.g., enalapril, benazepril), angiotensin receptor blockers (ARBs; e.g., telmisartan, losartan), managing of blood pressure (e.g., beta-blockers, calcium channel inhibitors), anticoagulants (e.g., clopidogrel), management of anemia (darbopoietin, varenzin, blood transfusion), management of hydration (fluid therapy), management of nausea and/or inappentance (omeprazole, famotidine, mirtazapine, maropitant), management of phosphorous load (phosphorous binders e.g., aluminum hydroxide), omega-3 fatty acid supplementation, or use of specially formulated diets for renal disease patients. In specific embodiments, the immunosuppressive therapy comprises glucocorticoids, cyclosporine, chlorambucil, mycophenolate.

In yet another aspect, the invention provides a method of treating a cat with glomerular or tubular damage comprising: obtaining a sample from the cat that comprises at least a first protein associated with glomerular or tubular damage; detecting the presence of the at least first protein associated with glomerular or tubular damage in the sample, wherein said protein comprises a molecular weight less than or equal to 40 kDa, a molecular weight between about 40 kDa and 70 kDa, or a molecular weight greater than or equal to 70 kDa; and treating the cat based on the presence of said protein in the sample. In certain embodiments, the detecting comprises separating the at least first protein associated with glomerular or tubular damage in the sample based on molecular weight.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. SDS-PAGE analysis of urine samples from dogs with MPGN and other glomerular diseases. Lanes 9 and 11 of the polyacrylamide gel shows the protein composition of urine samples from two different dogs with renal biopsy confirmed MPGN. Multiple proteins are observed having a molecular weight above 200 kDa and at least one band above 300 kDa. Sample identity, SDS-PAGE analysis details, and results of comprehensive renal tissue evaluation for each lane include: Mark 12 Unstained Standard, ranges from MW of 2.5 kDa to 200 kDa (Lanes 1 and 2); urine collected postmortem from a cat (Lane 3); urine collected postmortem from a Rottweiler (Lane 4); urine from a golden retriever, urine SDS-PAGE revealed moderate glomerular damage and mild tubular damage, comprehensive renal biopsy evaluation revealed chronic immune complex mediated glomerulonephritis with secondary focal segmental glomerulosclerosis and mild interstitial nephritis (Lanes 5 and 6); urine from a small mixed breed dog with juvenile onset of azotemia but not proteinuria, urine SDS-PAGE revealed a banding pattern similar to that from healthy dogs, comprehensive renal biopsy revealed renal maldevelopment (Lane 7); urine sample from the small mixed breed dog without normalizing to urine specific gravity (Lane 8); urine from a Boston terrier with marked proteinuria, urine SDS-PAGE revealed moderate to severe glomerular damage and moderate to possibly severe tubular damage, three protein bands are observed above 200 kDa, including one band above 300 kDa, the RSA200 was 1.53%, comprehensive renal biopsy revealed severe immune complex mediated mixed membranoproliferative glomerulonephritis with acute tubular necrosis, interstitial nephritis, tubular atrophy, and interstitial fibrosis (Lane 9); urine sample from a Pembroke Welsh corgi with asymptomatic proteinuria, urine SDS-PAGE revealed moderate glomerular and tubular damage, no protein bands were identified above 200 kDa, comprehensive renal biopsy was not performed to confirm the cause of glomerular and tubular damage (Lane 10); urine from a Collie dog with marked proteinuria and azotemia, urine SDS-PAGE revealed severe glomerular and tubular damage with 4 protein bands above 200 kDa including one protein band above 300 kDa, the RSA200 was 2%. Renal biopsy revealed severe immune complex mediated mixed membranoproliferative glomerulonephritis with segmental sclerosis and moderate interstitial nephritis (Lane 11); Mark 12 Unstained Standard, ranges from MW of 2.5 kDa to 200 kDa (Lane 12).

FIG. 2. SDS-PAGE analysis of urine samples from dogs with MPGN and other glomerular diseases. Lane 4 of the polyacrylamide gel shows the protein composition of a urine sample from a dog with renal biopsy confirmed MPGN. Multiple proteins are observed having a molecular weight above 200 kDa and at least one band above 300 kDa. Sample identity, SDS-PAGE analysis details, and results of comprehensive renal tissue evaluation for each lane include: Mark 12 Unstained Standard, ranges from MW of 2.5 kDa to 200 kDa (Lanes 1 and 2); urine from an intact male standard poodle with marked proteinuria, urine SDS-PAGE revealed mild to moderate glomerular damage, no protein bands were seen above 200 kDa, low molecular weight bands are consistent with prostatic proteins in this intact male, comprehensive renal biopsy revealed moderate to severe glomerular amyloidosis (Lane 3); urine from a mixed breed dog with marked proteinuria, urine SDS-PAGE revealed severe glomerular and tubular damage with 6 protein bands above 200 kDa including 3 protein bands above 300 kDa, the RSA200 is 3.3%, comprehensive renal biopsy revealed immune complex mediated membranoproliferative glomerulonephritis and acute interstitial nephritis (Lane 4); urine collected postmortem from an intact male Dalmatian with mild proteinuria; urine SDS-PAGE revealed primary tubular damage, comprehensive renal biopsy revealed renal maldevelopment and acute tubular necrosis (Lane 5); urine sample from the intact male Dalmatian with mild proteinuria without normalizing to urine specific gravity (Lane 6); urine from an Airedale terrier with marked proteinuria, urine SDS-PAGE revealed moderate glomerular damage and mild tubular damage, no bands were identified above 200 kDa, comprehensive renal biopsy revealed focal segmental glomerulosclerosis (Lane 7); urine collected postmortem from a Cavalier King Charles spaniel with marked proteinuria, urine SDS-PAGE revealed both glomerular and tubular damage, no bands were seen above 200 kDa, postmortem renal evaluation revealed acute tubular necrosis, marked podocyte injury, and focal segmental glomerulosclerosis (Lane 8); urine sample from an intact male Chihuahua, urine SDS-PAGE revealed severe glomerular damage and mild tubular damage, five protein bands were identified above 200 kDa including one protein band above 300 kDa (Lane 9); urine collected postmortem from a Yorkshire terrier, urine SDS-PAGE revealed mild to moderate glomerular damage and mild tubular damage, no bands were seen above 200 kDa, postmortem renal evaluation revealed mild interstitial nephritis, mild focal glomerular lipid emboli, and multifocal cystic dilated tubules (Lane 10); Mark 12 Unstained Standard, ranges from MW of 2.5 kDa to 200 kDa (Lanes 11 and 12).

FIG. 3. Receiver operating characteristic (ROC) curve for number of protein bands greater or equal to 200 kDa as a diagnostic marker for MPGN in dogs. The area under the curve (AUC) and its confidence interval are shown, along with a detailed report of the sensitivity and specificity analysis. Using a cut-point of ≥4 bands, specificity is 93% (CI: 86-99%) (AUC, 0.67; CI:0.56-0.79%) for MPGN.

FIG. 4. Receiver operating characteristic (ROC) curve for number of protein bands greater or equal to 300 kDa as a diagnostic marker for MPGN in dogs. The area under the curve (AUC) and its confidence interval are shown, along with a detailed report of the sensitivity and specificity analysis. Using a cut-point of ≥1 bands, specificity is 93% (CI: 86-99%) (AUC, 0.67; CI:0.56-0.75%) for MPGN.

FIGS. 5A-5B. Receiver operating characteristic (ROC) curve for RSA200 as a diagnostic marker for MPGN in dogs. FIG. 5A shows the area under the curve (AUC); and FIG. 5B shows a detailed report of the sensitivity and specificity analysis. Using a cut-point of 0.72, specificity is 93% (CI: 86-99%) for MPGN.

FIG. 6. Urine sample representing dogs with a spectrum of proteinuria levels using gel electrophoresis. Lanes 1 and 12: Molecular weight standards. Lane 9: Depicts a sample with HMW banding pattern most consistent with immune complex glomerulonephritis. The HMW banding patterns are denoted by the red box.

FIG. 7. Representative urine protein banding patterns in clinically healthy cats. Lanes 1 and 9: Molecular weight standard. Lanes 2-8: Urine samples from healthy, nonproteinuric cats. The red arrow denotes cauxin protein band. The black arrow denotes albumin band.

FIG. 8. Representative urine protein banding patterns in cats with pyelonephritis and/or urinary tract infections in increasing proteinuria. Lanes 2-7: Urine protein banding patterns from 6 cats with pyelonephritis and/or urinary tract infections in increasing proteinuria. Lanes 1 and 8: Molecular weight standard.

FIG. 9 Representative urine protein banding patterns in cats with biopsy confirmed primary tubular damage in increasing proteinuria. Lanes 2-5: Urine protein banding patterns in cats with biopsy confirmed primary tubular damage in increasing proteinuria. Lane 1: Molecular weight standard.

FIG. 10 Representative urine protein banding patterns in cats with biopsy confirmed primary glomerular damage. Lane 1 and 9: Molecular weight standard. Lanes 2-6: Urine samples from renal biopsy-confirmed ICGN in increasing proteinuria. Lanes 7 and 8: Urine samples from renal biopsy-confirmed non-ICGN in increasing proteinuria.

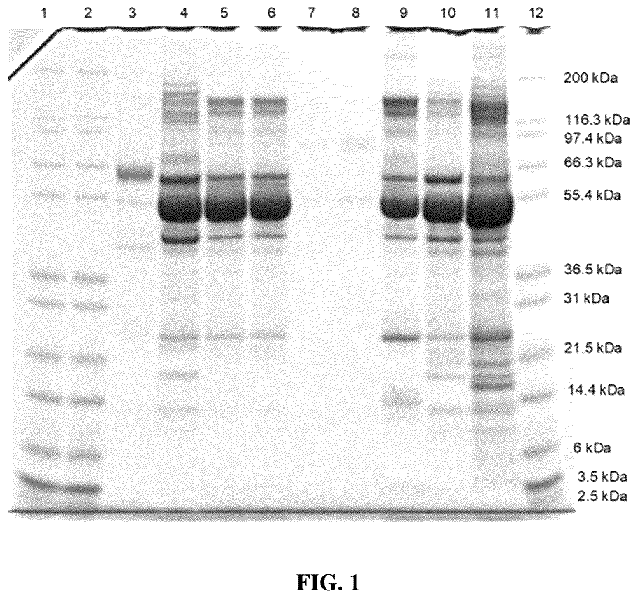

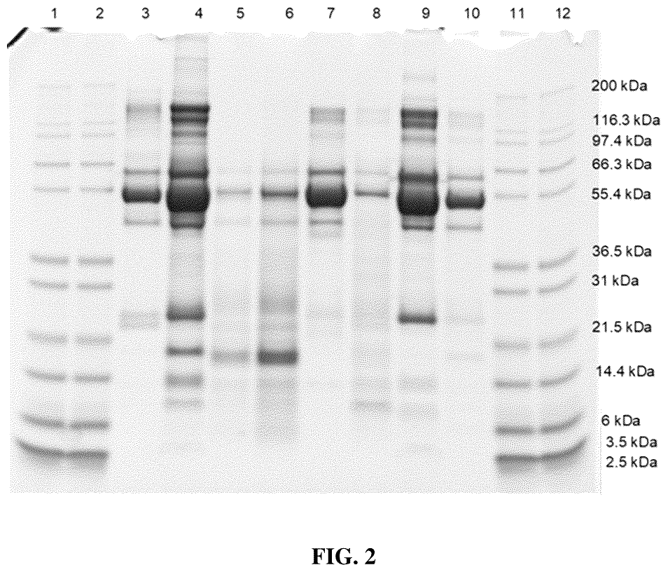

### DETAILED DESCRIPTION

Glomerular disease reduces the kidneys' ability to maintain a balance of certain substances in bloodstream. Normally, the kidneys filter toxins out of the bloodstream and excrete them in the urine but keep red blood cells and protein in the bloodstream. Glomerular disease is a common cause of chronic kidney disease (CKD) in dogs as well as cats. For example, previous studies have shown that pathologic evidence of kidney disease is common in dogs, and it is often due to glomerular disease (Müller-Peddinghaus & Trautwein 1977; Macdougall 1986). The three most common glomerular diseases in dogs with naturally occurring chronic kidney disease (CKD) are amyloidosis (AMYL), glomerulosclerosis (GS), and immune complex-mediated glomerulonephritis (ICGN). Data from the International Veterinary Renal Pathology Service (IVRPS) demonstrates that approximately half of dogs diagnosed with glomerular disease via a comprehensive renal evaluation have evidence of immune complexes (Schneider 2013; unpublished data). Based on this, ICGN is one of the most prevalent causes of glomerular disease in dogs. Furthermore, within the ICGN category are three major subcategories of disease: membranous glomerulonephritis (MGN), membranoproliferative glomerulonephritis (MPGN), and mesangioproliferative glomerulonephritis (MesGN).

Differentiation between ICGN and non-ICGN causes of glomerular disease is important due to differences in treatment strategies. Dogs and cats diagnosed with ICGN should be treated with immunomodulatory drugs in addition to standard therapy for proteinuria (RAAS blockade, omega-3 fatty acids, low-protein diet, and anticoagulant therapy). Immunosuppressive therapy is recommended when there is pathologic evidence of active immune pathogenesis, most compellingly identified by electron dense deposits or definitive positive staining by immunofluorescence on a kidney biopsy (Segev 2013). In particular, renal biopsy using light microscopy and advanced diagnostic modalities (transmission electron microscopy and immunofluorescence) is the current gold standard for determining the underlying pathology of glomerular disease in dogs and cats. Importantly, renal biopsy evaluation allows for the differentiation between ICGN and non-ICGN. Although renal biopsy procedures in dogs and cats are considered relatively safe, complications were reported in up to 13.4% of dogs in one study, (Vaden, S. L. et al. (2005) Journal of Veterinary Internal Medicine 19(6):794-801) with the most common complication being severe hemorrhage. Dogs developing complications in this study were more likely to have low body weight (≤5 kg) and severe azotemia (serum creatinine ≥5). Therefore, the potential clinical benefits of information gained from a renal biopsy must be weighed against the potential for complication for each individual patient. In addition to the risks of performing a renal biopsy in at-risk patients, the expense associated with the procedure can be cost-prohibitive.

Clinicians often must make a clinical judgment whether or not to treat with immunosuppressive therapy in the absence of a kidney biopsy, but immunosuppressive therapy is contraindicated in non-ICGN glomerular diagnostic categories. Specifically, immunosuppressive therapy in non-ICGN glomerular disease can lead to gastrointestinal signs (e.g., anorexia, nausea, vomiting, diarrhea), bone marrow suppression, and secondary infections.

A non-invasive method for identifying ICGN from non-ICGN causes of glomerular disease, including the major subcategories of ICGN such as membranoproliferative glomerulonephritis (MPGN), would help ensure the correct treatment as well as avoid the detrimental effects of contraindicated therapies in non-ICGN glomerular diseases. Moreover, the ability to non-invasively identify MPGN or other ICGN related glomerular disease could allow patients that are poor candidates for renal biopsy to be accurately diagnosed and treated effectively for the first time. Similarly, methods to non-invasively identify glomerular or tubular damage in cats could allow patients to be accurately diagnosed and treated effectively if a more invasive confirmatory renal biopsy is not possible.

Excluding renal biopsy, there are currently no commercially available diagnostic tests for MPGN to effectively inform treatment decisions. Determination of the number and/or optical density (i.e. relative surface area) of urine protein bands larger than 200 kDa via SDS-PAGE is a minimally invasive technique that is specific for detection of MPGN. Moreover, SDS-PAGE analysis is significantly faster than a comprehensive renal biopsy evaluation and is significantly less expensive (approximately 10-15% of the cost of comprehensive renal biopsy evaluation, not including the added expenses of tissue collection and anesthesia). The detection of proteins larger than 200 kDa in a urine sample thus serves as a novel means of MPGN detection. Moreover, the present disclosure provides methods of using such detection to directly and accurately inform treatment decisions in canine or feline glomerular disease.

The present disclosure therefore represents a significant advance in the art in that it provides a means to differentiate between diagnostic categories of glomerular disease, and thus effectively treat such diseases. In particular, the present disclosure describes how the presence of multiple proteins greater than or equal to 200 kDa in a canine urine sample was observed in dogs with MPGN compared to control dogs as well as dogs with other glomerular disease types. Thus, detecting the presence of at least two proteins in the sample having a molecular weight greater than or equal to 200 kDa represents a non-invasive biomarker to identify dogs for treatment with immunosuppressive therapy in the absence of a renal biopsy. Furthermore, the present disclosure also describes how detecting the presence of at least one protein in the sample having a molecular weight greater than or equal to 300 kDa; or an RSA200 value greater than or equal to 0.7% in the sample represents a non-invasive biomarker to identify dogs for treatment with immunosuppressive therapy in the absence of a renal biopsy. The methods and compositions disclosed herein offer the opportunity to identify and effectively treat patients to improve and prolong quality of life without the need for a comprehensive renal biopsy.

Additionally, the present disclosure also represents a significant advance in the art in that it provides a means to differentiate between normal, healthy cats and cats experiencing glomerular or tubular damage, allowing clinicians to effectively treat such maladies. In particular, the present disclosure describes how the presence of multiple proteins having a molecular weight less than or equal to 40 kDa, a molecular weight between about 40 kDa and 70 kDa, or a molecular weight greater than or equal to 70 kDa in a feline urine sample was observed in cats with glomerular or tubular damage compared to control cats. Thus, detecting the presence of at least two proteins in the sample having a molecular weight less than or equal to 40 kDa, at least four proteins in the sample having a molecular weight between about 40 kDa and 70 kDa, or at least two proteins in the sample having a molecular weight greater than or equal to 70 kDa represents a non-invasive biomarker to identify cats for treatment in the absence of a renal biopsy. The methods and compositions disclosed herein offer the opportunity to identify and effectively treat patients to improve and prolong quality of life without the need for a comprehensive renal biopsy.

Accordingly, provided herein are methods and compositions for identifying MPGN, primarily in dogs, that may be used to treat patients with the most effective therapy without relying on potentially incorrect assumptions. In some embodiments, the methods described herein are useful in the evaluation of a patient, for example, for evaluating diagnosis, prognosis, and response to treatment. In other aspects, the present invention comprises evaluating a dog with glomerular disease. In some embodiments, the evaluation may be selected from diagnosis, prognosis, and response to treatment. Similarly, provided herein are methods and compositions for identifying glomerular or tubular damage in cats that may be used to treat patients with the most effective therapy without relying on potentially incorrect assumptions. In some embodiments, the methods described herein are useful in the evaluation of a patient, for example, for evaluating diagnosis, prognosis, and response to treatment. In other aspects, the present invention comprises evaluating a cat with glomerular disease. In some embodiments, the evaluation may be selected from diagnosis, prognosis, and response to treatment.

Diagnosis refers to the process of attempting to determine or identify a possible disease or condition, such as, for example, a glomerular disease (e.g. ICGN, AMYL, and GS) or specific category thereof (e.g. MGN, MPGN, and MesGN). Prognosis refers to predicting a likely outcome of a disease or condition, such as, for example, a glomerular disease or specific category thereof. A complete prognosis often includes the expected duration, the function, and a description of the course of the disease, such as progressive decline, intermittent crisis, or sudden, unpredictable crisis. Response to treatment is a prediction of a patient's medical outcome when receiving a treatment. Responses to treatment can be, by way of non-limiting example, improved renal function (e.g., decreased proteinuria, increased renal filtration), survival, and mitigation of proteinuric complications (e.g. hypercoagulability, venous thromboembolism, ascites).

The term “treating a glomerular disease” refers to ameliorating the effects of, or delaying, halting, or reversing the progress of, a glomerular disease as defined herein. In some embodiments, treating the glomerular disease can refer to the selection and administration of the appropriate therapy based on the type of glomerular disease present in the patient. For example, appropriate therapies for patients diagnosed with glomerular disease (including non-ICGN glomerular disease) include, e.g. standard renoprotective therapies such as angiotensin-converting enzyme (ACE) inhibitors (e.g., enalapril, benazepril, captopril, ramipril), angiotensin receptor blockers (ARBs; e.g., telmisartan, losartan), management of blood pressure (e.g., beta-blockers, calcium channel inhibitors), anticoagulants (e.g., aspirin, clopidogrel, rivaroxaban), omega-3 fatty acid supplementation, and specially formulated diets for renal disease patients (See, J Vet Intern Med. 2013. Nov-Dec; 27 Suppl 1:S27-43). Additionally, appropriate therapies for patients diagnosed with MPGN or other ICGN disease types include administration of mycophenolate, cyclophosphamide, azathioprine, chlorambucil, and cyclosporine.

“Patient” as used herein includes, e.g., a dog or a cat. In preferred embodiments, the patient is a dog. A “dog” may include, e.g., a Labrador Retriever, Golden Retriever, German Shepherd, Beagle, French Bulldog, Bulldog, Poodle, Yorkshire Terrier, Boxer, Rottweiler, Pembroke Welsh Corgi, Cavalier King Charles Spaniel, Dachshund, Dobermann, Shih Tzu, Australian Shepherd, Boston Terrier, German Shorthaired Pointer, Great Dane, Pomeranian, Bernese Mountain Dog, Siberian Husky, Miniature Schnauzer, a Border Collie, or a combination thereof (i.e. mixed breeds).

As used herein, the term “control” dog (or likewise a “control” cat) refers to an appropriate dog or cat that is used for comparison to a patient. In some embodiments, a control dog lacks glomerular disease. In other embodiments, a control dog comprises a glomerular disease type different than the patient. For example, in specific embodiments a control dog may comprise amyloidosis or glomerulosclerosis, whereas the patient comprises immune complex-mediated glomerulonephritis.

In some embodiments, the present methods direct a clinical decision regarding whether a patient is to receive a specific treatment. In one embodiment, the present methods are predictive of a positive response to immunosuppressive therapy. In certain embodiments, the present invention directs the treatment of a glomerular disease patient, including, for example, what type of treatment should be administered or withheld. For example, a patient that is shown to have at least two proteins having a molecular weight greater than or equal to 200 kDa in a urine sample may receive such treatment as immunosuppressive therapy. In some embodiments, a patient that is shown to have at least one protein having a molecular weight greater than or equal to 300 kDa in a urine sample may also receive such treatment as immunosuppressive therapy. In still other embodiments, a patient that is shown to have an RSA200 value greater than or equal to 0.7% in the sample in a urine sample may similarly receive such treatment as immunosuppressive therapy.

In one embodiment, the present methods may indicate that a patient will not be or will be less responsive to a specific treatment and therefore such a patient may not receive such treatment as immunosuppressive therapy. Accordingly, in some embodiments, the present methods provide for providing or withholding immunosuppressive therapy according to a patient's likely response. In this way, a patient's quality of life may be improved and the cost of care may be reduced.

In some embodiments, the present methods direct a clinical decision regarding whether a patient is to receive a specific type of treatment. Accordingly, in some embodiments, the present methods are a guiding test for patient treatment. Furthermore, the present methods provide information about the likely response that a patient is to have to a particular treatment. In some embodiments, the present methods provide a high likelihood of response and may direct treatment. In some embodiments, the present methods provide a low likelihood of response and may direct cessation of treatment or avoidance of specific treatments, including immunosuppressive therapy, and the use of alternative renal protective therapies, to avoid unnecessary negative effects from contraindicated therapies for a better quality of life.

As used herein, the term “severity of glomerular disease” refers to a qualitative or quantitative assessment of the level of advancement of a glomerular disease. Criteria used to determine the stage of a glomerular disease includes, but is not limited to, the number of detectable polypeptides having a molecular weight greater than or equal to 200 kDa associated with glomerular disease in a urine sample from a patient.

As used herein, the term “polypeptide” refers to a chain of at least two covalently linked amino acids. An example of a polypeptide is a protein. Polypeptides can be separated and/or purified from natural sources (e.g., a biological sample) by known methods such as DEAE ion exchange, gel filtration, and hydroxyapatite chromatography. The composition of polypeptides (i.e. proteins) or the presence of one or more proteins of interest in a sample can also be measured using any appropriate method, e.g., column chromatography, polyacrylamide gel electrophoresis, or HPLC analysis. In the context of protein electrophoresis, proteins may also be referred to as protein bands.

The term “level” or “concentration” refers, e.g., to a determined abundance or relative abundance of a polypeptide of interest. The term “relative surface area” (RSA) refers to a determined abundance of one or more polypeptides compared to an appropriate reference, e.g. from a computed average abundance value in SDS-PAGE analyses. In particular, “relative surface area” of a single protein band is the proportion of its optical density compared to the overall number and optical density of all protein bands in a patient urine sample, e.g. when analyzed using SDS-PAGE. Optical density equates to darkness or intensity of a protein band and reflects abundance of a protein in a sample. A pattern is not limited to the comparison of two polypeptides but is also related to multiple comparisons to reference polypeptides or samples. For example, the term “RSA200” refers to the optical density of proteins larger than 200 kDa compared to the overall optical density of all protein bands in a patient sample such as a urine sample. Similarly, the term “RSA70” refers to the optical density of proteins larger than 70 kDa compared to the overall optical density of all protein bands in a patient sample; the term “RSA40-70” refers to the optical density of proteins between about 40 kDa and 70 kDa compared to the overall optical density of all protein bands in a patient sample; and the term “RSA40” refers to the optical density of proteins less than or equal to 40 kDa compared to the overall optical density of all protein bands in a patient sample, such as a urine sample. In embodiments wherein the relative surface area of the protein is quantified, the RSA value (e.g. RSA200, RSA70, RSA40-70, or RSA40) can be referred to as greater than or equal to 0.01%, 0.02%, 0.025%, 0.05%, 0.075%, 0.1%, 0.2%, 0.3%, 0.4%, or 0.5%.

Detection (e.g., of an amplification product, of a hybridization complex, of a polypeptide) can be accomplished using detectable labels that may be attached or associated with a hybridization probe or antibody. The term “label” is intended to encompass the use of direct labels as well as indirect labels. Detectable labels include enzymes, prosthetic groups, fluorescent materials, luminescent materials, bioluminescent materials, radioactive materials, and protein-specific staining reagents. The detection of proteins above background levels in a sample can be through any methodologies known to those skilled in the art of molecular biology. Examples of protein detection methodologies include, but are not limited to, enzymatic assays for detecting enzyme activity of polypeptides, protein gel electrophoresis, Western blots, immunoprecipitation, and enzyme-linked immunoassays to detect polypeptides. Other techniques such as in situ hybridization, protein staining, and immunostaining also can be used to detect the presence or expression of polypeptides and/or polynucleotides. Methods for performing all of the referenced techniques are known in the art.

In some embodiments, detecting comprises performing gel electrophoresis (e.g. sodium dodecyl-sulfate polyacrylamide gel electrophoresis; SDS-PAGE) on a sample. The term “electrophoresis gel”, as used herein, refers to a gel used for electrophoretic separation of a sample. This technique involves the separation of proteins based on their size. In brief, proteins present in samples heated under denaturing and/or reducing conditions become unfolded and coated with SDS detergent molecules, acquiring a high net negative charge that is proportional to the length of the polypeptide chain. When loaded onto a gel matrix and placed in an electric field, the negatively charged protein molecules migrate towards the positively charged electrode and are separated by a molecular sieving effect.

SDS-PAGE is a useful method for determining the molecular weight (MW) of a protein in a sample, since the migration rate of a protein coated with SDS is inversely proportional to the logarithm of its molecular weight. In the presence of sodium dodecyl sulfate (SDS), proteins form SDS-protein complexes that are negatively charged. As a result, the non-covalent interactions between the proteins are cleaved. Therefore, SDS breaks the hydrophobic interactions and hydrogen bonds.

Many SDS-PAGE protocols are known in the art. The key to accurate MW determination is selecting separation conditions that produce a linear relationship between log MW and migration within the likely MW range of the protein of interest, such as proteins having a molecular weight greater than or equal to 200 kDa. SDS-PAGE may be further categorized, e.g. as nonreducing or reducing SDS-PAGE depending on the presence of a denaturing reagent such as dithiothreitol (DTT) or mercaptoethanol, which reduces any disulfide bonds present in the protein(s). The methods provided herein may comprise SDS-PAGE under nonreducing or under reducing conditions. It is understood that certain SDS-PAGE conditions may affect the mobility of a protein in a polyacrylamide gel. For example, in the absence of a reducing reagent agent disulfide bonds may still be present in the protein(s). Such secondary structures may impact protein migration. Other factors that may affect electrophoretic migration include posttranslational modification and amino acid compositions (e.g. highly acidic or basic proteins, or proteins with high proline content). As described herein proteins having a high molecular weight, for example, greater than or equal to 200 kDa or 300 kDa (≤40 kDa, 40-70 kDa, or ≥70 kDa in the case of cats) include proteins that migrate at the same rate or a slower rate when compared to an appropriate molecular weight standard (e.g. Mark12 Unstained Standard; Invitrogen Life Technologies) under non-reducing conditions. In some embodiments, the presence of such proteins may be further described as detectable by SDS-PAGE using a conventional protein gel staining reagent; visually detectable following conventional protein gel staining; or detectable above background levels on a polyacrylamide gel when using a conventional protein gel staining reagent. These protein bands visible on the polyacrylamide gel may include one or more distinct proteins, each of which migrates at the same rate during SDS-PAGE. In such embodiments, the methods provided herein comprise detecting in a sample from said dog the presence of at least two protein bands in the sample having a molecular weight greater than or equal to 200 kDa; at least one protein band in the sample having a molecular weight greater than or equal to 300 kDa; or an RSA200 value greater than or equal to 0.7% in the sample. In this regard, the present disclosure provides for the first time that ability of protein bands larger than 200 kDa to identify MPGN in a dog with high specificity and directly inform treatment decisions based on the same. Similarly, the present disclosure provides for the first time that ability of protein bands less than or equal to 40 kDa, between about 40 kDa and 70 kDa, greater than or equal to 70 kDa, in the sample to identify glomerular or tubular damage in a cat and directly inform treatment decisions based on the same.

Additional conditions that may be varied when performing SDS-PAGE include, but are not limited to voltage, current, and time. Voltage (V) is the difference in electrical potentials between two charges and is the primary parameter for defining the speed that the protein will move through a gel during SDS-PAGE; and current (I) refers to the flow of electric charge past a point in a circuit. In some embodiments, e.g., the methods provided herein comprise performing sodium dodecyl-sulfate polyacrylamide gel electrophoresis using a polyacrylamide gel having Bis-Tris from about 4% to about 20%, from about 4% to about 16%, or from about 4% to about 12%. SDS-PAGE is commonly conducted at a constant voltage, e.g. approximately between 100V-300V. SDS-PAGE may also be conducted at a constant current, e.g. approximately between 10-20 mA. In some embodiments, for example, the methods provided herein comprise performing non-reducing sodium dodecyl-sulfate polyacrylamide gel electrophoresis on a 4-12% Bis-Tris polyacrylamide gel at 200 V for 35 minutes.

Visualization of protein bands following SDS-PAGE is carried out by incubating the gel with a staining solution. The commonly used methods are Coomassie and silver staining. Silver staining is a more sensitive staining method than Coomassie staining. Coomassie staining, though less sensitive, is quantitative and Coomassie-stained proteins can be used for downstream applications. Polyacrylamide gels can be stained with dye from about 1 h to about 24 h, e.g. for 2 h; and excess stain may be washed in an appropriate solution (e.g. ultra-pure water) to remove non-specific staining. Many protocols for protein gel staining are known in the art. As one example, Imperial Protein Stain (ThermoFisher Scientific, Rockford, IL) is a ready-to-use colorimetric stain formulated with coomassie dye R-250 that delivers consistent nanogram-level detection of proteins in polyacrylamide electrophoresis gels or nitrocellulose membranes. In certain embodiments, the methods described herein comprise detecting in a sample from said dog the presence of proteins having a molecular weight greater than or equal to 200 kDa above background levels. For example, above background levels that would be detected in an animal without disease or an animal without MPGN. In one embodiment, for example, this can be detected by SDS-PAGE using a conventional dye, e.g. coomassie dye.

After visualization by a protein-specific staining technique (e.g. Coomassie blue dye, Imperial Protein Stain; ThermoFisher Scientific, Rockford, IL), the size of a protein can be estimated by comparison of its migration distance with that of a standard of known molecular weight directly or with the assistance of gel imaging software. In certain embodiments, the separated proteins can be transferred onto a positively charged membrane and probed with protein-specific antibodies.

In some embodiments, the presence of a protein can be further described as detectable by SDS-PAGE using a conventional protein gel staining reagent, e.g. a coomassie dye. The presence of a protein can also be described as visually detectable following conventional protein gel staining; or detectable above background levels on a polyacrylamide gel when using a conventional protein gel staining reagent (e.g. about ≥3 ng/μL).

The present invention provides a novel method of detecting membranoproliferative immune complex-mediated glomerulonephritis (MPGN) in a dog or cat comprising detecting in a sample from said dog or cat the presence of at least two proteins in the sample having a molecular weight greater than or equal to 200 kDa; at least one protein in the sample having a molecular weight greater than or equal to 300 kDa; or an RSA200 value greater than or equal to 0.7% in the sample, wherein said detecting is indicative of MPGN in the dog or cat. Such proteins present unique information, which may be used to determine the type of glomerular disease present. Such information can directly inform proper treatment of glomerular disease in dogs and cats absent a renal biopsy and comprehensive pathological evaluation.

According to some embodiments, the method comprises detecting the presence of at least three, four, five, six, or seven proteins in the sample having a molecular weight greater than or equal to 200 kDa. According to other embodiments, the method comprises detecting the presence of at least two proteins in the sample having a molecular weight greater than or equal to 300 kDa. In other embodiments, especially with respect to cats, the methods described herein comprise detecting the presence of at least two, three, four, five, six, seven, or more proteins in the sample having a molecular weight less than or equal to 40 kDa, between about 40 kDa and 70 kDa, and/or greater than or equal to 70 kDa In some embodiments, the concentration of said protein(s) in the sample is at least 2.5 ng/μL, 3 ng/μL, 3.5 ng/μL, 4 ng/μL, 4.5 ng/μL, or 5 ng/μL.

The term “marker” or “biomarker” refers to a biological molecule, e.g., a protein, whose presence or concentration can be detected and correlated with a known condition, such as a disease state, or with a clinical outcome, such as response to a treatment. In particular, the present disclosure provides a method of detecting membranoproliferative immune complex-mediated glomerulonephritis (MPGN) in a dog comprising obtaining a sample from the dog that comprises at least a first protein associated with MPGN; detecting the presence of at least two proteins in the sample having a molecular weight greater than or equal to 200 kDa; at least one protein in the sample having a molecular weight greater than or equal to 300 kDa; or an RSA200 value greater than or equal to 0.7% in the sample, wherein said detecting is indicative of MPGN in the dog.

The present disclosure also provides method of treating a dog with membranoproliferative immune complex-mediated glomerulonephritis (MPGN) comprising: a) obtaining a sample from the dog that comprises at least a first protein associated with MPGN; b) detecting the presence of the at least first protein associated with MPGN in the sample, wherein said protein comprises a molecular weight greater than or equal to 200 kDa; and (c) treating the dog based on the presence of said protein in the sample. In further embodiments, said detecting comprises separating the at least first protein associated with MPGN in the sample based on molecular weight.

Kits for performing the methods of the invention are also provided, said kit comprising means for determining in a sample from a patient, e.g. the presence of at least two proteins in the sample having a molecular weight greater than or equal to 200 kDa; at least one protein in the sample having a molecular weight greater than or equal to 300 kDa; or an RSA200 value greater than or equal to 0.7% in the sample. Alternatively, said kit may comprise means for determining in a sample from a patient, the presence of at least two, three, four, five, six, seven, or more proteins in the sample having a molecular weight less than or equal to 40 kDa, between about 40 kDa and 70 kDa, or greater than or equal to 70 kDa. The means for determining the presence of said protein(s) may comprise reagents for performing protein gel electrophoresis or any other suitable means for determining the presence of a protein of interest. The kit, in certain embodiments, may also comprise reagents for quantification of the protein of interest.

All the essential materials and/or reagents required for detecting MPGN (or glomerular or tubular damage in cats) associated proteins in a sample may be assembled together in such a kit. Also included may be enzymes or other reagents suitable for detection and/or quantification, and, for instance, buffers to provide the necessary reaction conditions. Such kits generally will comprise, in suitable means, distinct containers for any reagent or enzyme, or for performing a given step in the contemplated assay.

As used herein, the term “sequence identity” refers to the extent to which two optimally aligned polynucleotide sequences or two optimally aligned polypeptide sequences are identical. An optimal sequence alignment is created by manually aligning two sequences, e.g. a reference sequence and another sequence, to maximize the number of amino acid matches in the sequence alignment with appropriate internal amino acid insertions, deletions, or gaps. As used herein, the term “reference sequence” may refer to a sequence of a protein indicative of MPGN in a dog as described herein.

As used herein, the term “percent sequence identity” or “percent identity” or “% identity” is the identity fraction times 100. The “identity fraction” for a sequence optimally aligned with a reference sequence is the number of nucleotide matches in the optimal alignment, divided by the total number of nucleotides in the reference sequence, e.g. the total number of nucleotides in the full length of the entire reference sequence. Thus, one embodiment of the invention is a polypeptide molecule comprising a sequence that when optimally aligned to a reference sequence, has at least about 85 percent identity, at least about 90 percent identity, at least about 95 percent identity, at least about 96 percent identity, at least about 97 percent identity, at least about 98 percent identity, or at least about 99 percent identity to the reference sequence. In particular embodiments such sequences may be defined as being indicative of glomerular disease in a patient.

### EXAMPLES

The following examples are included to demonstrate preferred embodiments of the invention. It should be appreciated by those of skill in the art that the techniques disclosed in the examples, which follow represent techniques discovered by the inventor to function well in the practice of the invention, and thus can be considered to constitute preferred modes for its practice. However, those of skill in the art should, in light of the present disclosure, appreciate that many changes can be made in the specific embodiments, which are disclosed and still obtain a like or similar result without departing from the spirit and scope of the invention.

### Example 1

Materials and Methods for Investigating Proteins Associated with MPGN in a Urine Sample

### Sample Collection and Processing

In the retrospective studies described herein, whole urine samples collected from dogs undergoing renal biopsy for suspicion of glomerular disease and submitted to the International Veterinary Renal Pathology Service (IVRPS) for diagnostic purposes between April 2017 to December 2020 were evaluated. All samples were shipped on ice and were typically received and processed within 24 hours of collection.

Upon receipt, macroscopic evaluation of whole urine was performed, including test strip and urine specific gravity evaluation. Remaining urine was centrifuged at 450 g for 5 minutes. Supernatant (less 20% volume remaining with sediment) was removed, aliquoted, and stored at −80 degrees Celsius until analysis via gel electrophoresis (less than 2 weeks following sample receipt). Urine sediment was resuspended in the remaining 20% volume of supernatant. Microscopic evaluation of urine sediment included semiquantitative enumeration of formed elements within the urine (e.g. RBCs, WBCs, epithelial cells, casts, crystals, bacteria, and lipids). Cases with pyuria (>5 WBCs per high powered field) or bacteriuria were excluded from further analysis. Based on previous studies, cases with microscopic hematuria were included. Blood contamination of ≥0.125% increases UPC; however, if the urine remains yellow (microscopic hematuria only, without macroscopic hematuria), then there is negligible chance that a UPC >0.5 will be solely due to the added blood.

### Histopathologic Analysis

Renal biopsies were processed routinely for light microscopy (LM), transmission electron microscopy (TEM), and immunofluorescence (IF) as previously described (e.g. Schneider et. al, 2013). In brief, specimens for LM were sectioned at 3 μm thickness and stained with H&E, Masson's Trichrome (MT), Periodic acid-Schiff (PAS), and Jones' methenamine silver (JMS). Congo red (CR) staining was performed on specimens sectioned at 8-10 μm thickness if amyloid was suspected on LM. TEM was not always performed in cases with amyloidosis diagnosed by LM and CR staining (i.e. typical apple-green birefringence when viewed under polarized light). For TEM, tissues were fixed in chilled 3% buffered glutaraldehyde. Specimens were postfixed in 1% osmium tetroxide, dehydrated in a series of graduated alcohols, infiltrated in an acetone/epoxy plastic, and embedded in a plastic mold. Plastic blocks were cut with an ultramicrotome. Thick sections were stained with toluidine blue. Sections then were evaluated, and appropriate areas identified for thin sectioning. Thin sections were cut at silver-grey interference color (55-60 nm) and placed on slotted copper grids coated with Formvar. Grids were stained with uranyl acetate and lead citrate and were examined in a transmission electron microscope. TEM specimens initially were evaluated by a single nephropathologist at The Ohio State University. Unfixed tissue samples for IF evaluation were embedded in Optimal Cutting Temperature medium and snap frozen on dry ice. After cryosectioning at 5 μm thickness, sections were labeled with an appropriate dilution of fluorescein isothiocyanate-labeled anti-IgG, anti-IgM, anti-IgA, anti-C3, and anti-lambda light chains antibodies. All slides were examined by a single nephropathologist with an epifluorescence microscope using appropriate filters.

Comprehensive evaluation of renal biopsies included: LM evaluation of biopsies stained with hemotoxylin and eosin, periodic acid Schiff, Masson's trichrome, and Jones' methanamine silver; TEM for assessment of glomerular basement membrane structure, presence and location of electron dense deposits, and identification and characterization of abnormal fibrils; and IF for detection of positive C3, IgA, IgG, IgM, and/or LLC antibody labeling of immune deposits in glomeruli. Glomerular disease diagnoses were categorized into non-ICGN glomerular diseases (amyloidosis and focal segmental glomerulosclerosis (FSGS)) and ICGN glomerular diseases (including MGN, mixed MGN, MPGN, mixed MPGN, and MesGN). Additionally, dogs were also categorized based on a diagnosis of MPGN versus non-MPGN (which included any non-MPGN disease, regardless if immune complex mediated or not).

### Gel Electrophoresis of Urine Supernatant

Nonreducing, denaturing gel electrophoresis was performed on all urine samples, using precast 12-well, 4%-12% Bis-Tris gels (Bolt 4-12% Bis-Tris Gel and Invitrogen Mini Gel Tank; Life Technology Corporation, Carlsbad, CA). Urine samples were diluted as previously described with ultrapure water based on the USG to a volume of 30 μL (urine volume (in microliters)=0.065/(USG-1); water volume (in microliters)=30 ul−urine volume (ul)), followed by addition of 10 μL lithium dodecyl sulfate (LDS) sample buffer (Bolt LDS Sample Buffer [4×]; Invitrogen Life Technologies, Carlsbad, CA) for a final volume of 40 μL. Samples were heated at 70° C. for 10 minutes. 20 μL of diluted urine sample or 5 μL of a molecular weight standard (Mark12 Unstained Standard; Invitrogen Life Technologies) were loaded into separate gel lanes. Gels were run in duplicate (200V for 35 minutes) in SDS buffer (Bolt MES SDS Running Buffer [20×]; Invitrogen Life Technologies). Following water washes, gels were stained (Imperial Protein Stain; ThermoFisher Scientific, Rockford, IL) for 2 hours, washed and destained overnight with ultrapure water. Gels were photographed (Gel Doc XR+System; Bio-Rad Laboratories Inc., Hercules, CA), placed in a solution of 2% glycerol/20% ethanol for 20 minutes, and dried between cellulose sheets.

### Analysis of Gels

Digital photographs of the gels were uploaded to commercially available software (GelComparII; Applied Maths, Austin, TX). Using these images and the dried gels, protein bands were manually identified in each lane (each lane typically corresponded to a different patient sample). The total number and molecular weights of the protein bands in each sample were determined. Additionally, the relative surface area of each protein band within each sample was automatically determined in the GelComparII software. Relative surface area (RSA) of a single protein band is the proportion of its optical density compared to the overall number and optical density of all protein bands in a patient urine sample. Optical density equates to darkness or intensity of a protein band and reflects abundance of a protein in a sample. Using this information, the RSA of all protein bands greater than 200 kDa could be calculated. This was termed RSA200 and is the proportion of overall number and optical density (abundance) of all proteins in a sample comprised of those proteins larger than 200 kDa.

### Example 2

### Analysis and Identification of Diagnostic Markers for MPGN

### Statistical Analysis

Statistical analysis was performed using StataBE 17.0 (Stata Corp. LP, College Station, TX). Mean, median and range of the number of urine proteins above 200 kDa and RSA200 were determined for ICGN and non-ICGN categories and MPGN and non-MPGN categories. Wilcoxon rank sum analysis was used to determine significant differences in number of bands above 200 kDa, RSA200, and UPC between ICGN vs. non-ICGN dogs and between MPGN vs. non-MPGN dogs. Receiver operator characteristic (ROC) analysis was used to determine the optimal cut-point for the ability of number of bands above 200 kDa, RSA200, and UPC to distinguish ICGN from non-ICGN and MPGN from non-MPGN. Using these optimal cut-points, sensitivity and specificity were determined for the ability of number of bands above 200 kDa, RSA200, and UPC to distinguish between canine ICGN and non-ICGN and between MPGN and non-MPGN.

Ninety-eight dogs, ranging in age from 1 year and 11 months to 16 years and 3 months, were included in the final analysis (Table 1). Overrepresented breeds included large mixed breed dogs (n=14, 14.3%), small mixed breed dogs (n=10, 10.2%), Labrador Retrievers, (n=5, 5.1%), Wire Fox Terrier (n=4, 4.1%), Yorkshire Terriers (n=3, 3.1%), and Maltese (n=3, 3.1%). Table 1 provides a descriptive summary of selected clinicopathologic data (UPC, SCr, sAlb, BP), age, and sex for each specific glomerular disease category as identified on comprehensive renal biopsy (amyloidosis, FSGS, MGN/mixed MGN, and MPGN/mixed MPGN). The number of bands above 200 kDa and RSA200 were both significantly greater in MPGN compared with non-MPGN dogs; these values did not differ significantly between ICGN and non-ICGN dogs (Table 2). Additionally, UPC did not differ significantly between ICGN and non-ICGN dogs nor between MPGN or non-MPGN dogs.

TABLE 1
Table of urine and serum biochemical abnormalities of dogs biopsied
for evaluation of suspected glomerular disease, broken dogs
by histopathologic diagnosis. Reported as median (range)

| | | | | | | | Non-invasive |
|---|---|---|---|---|---|---|---|
| | | | | | | | blood |
| | | | | | Creatinine | Albumin | pressure |
| Specific | | | Age (years) | UPC | (mg/dL) | (g/dL) | (mmHg) |
| Glomerular | | | Median | Median | Median | Median | Median |
| Disease | | | (range); | (range); | (range); | (range); | (range); |
| Category | N | Sex | n = 97 | n = 91 | n = 88 | n = 88 | n = 79 |
| Amyloid | 13 | M: 1 | 8 (2- | 10.4 (3.4- | 1.25 (0.75- | 1.85 (1.3- | 152.5 (110- |
| | | MN: 5 | 16) | 41.1) | 3.6) | 2.8) | 200) |
| | | F: 0 | | | | | |
| | | FS: 7 | | | | | |
| FSGS | 29 | M: 2 | 7 (2- | 7.2 (1.68- | 1.05 (0.3- | 2.9 (1.5- | 160 (120- |
| | | MN: 10 | 13) | 20.6) | 3.5) | 4.1) | 200) |
| | | F: 1 | | | | | |
| | | FS: 16 | | | | | |
| Mesangiopro | 11 | M: 1 | 7 (1- | 4.8 (3.6- | 1.19 (0.5- | 2.55 (1.5- | 153 (130- |
| liferative GN | | MN: 3 | 11) | 15.5) | 4.3) | 3.4) | 180) |
| | | F: 0 | | | | | |
| | | FS: 7 | | | | | |
| MGN and | 15 | M: 0 | 6 (2- | 7.8 (0.3- | 1.3 (0.64- | 1.9 (1.2- | 152.5 (110- |
| Mixed MGN | | MN: 6 | 12) | 9.8) | 3.4) | 3.4) | 180) |
| | | F: 0 | | | | | |
| | | FS: 9 | | | | | |
| MPGN and | 30 | M: 1 | 6 (1- | 8.35 (2- | 2.45 (0.3- | 2.1 (1- | 150 (85- |
| Mixed | | MN: 9 | 13) | 17.8) | 5.6) | 2.8) | 220) |
| MPGN | | F: 2 | | | | | |
| | | FS: 18 | | | | | |

UPC, urine protein:creatinine ratio;
FSGS, focal segmental glomerulosclerosis;
GN, glomerulonephritis;
MGN, membranous glomerulonephropathy;
MPGN, membranoproliferative glomerulonephritis

TABLE 2
Frequency, median and range of number of bands >200 kDa and relative surface area of urine protein
bands >200 kDA (rSA200, in greyscale units) and urine protein to creatinine ratio (UPC) from dogs
with glomerular disease. Comparing MPGN, membranoproliferative glomerulonephritis, ICGN
immune complex glomerulonephritis

| General Glomerular | Frequency | UPC, Median | # of Bands >200 kDa, | RSA >200 (%), |
|---|---|---|---|---|
| Disease Category | (%) | (range) | Median (Range) | Median (range) |
| Non-MPGN | 69 | 6.8 (0.3-41.4) | 0 (0-6) | 0.0 (0.0-3.2) |
| MPGN | 31 | 8.4 (2.0-9.8) | 2 (0-7)* | 0.3 (0.0-7.8)* |
| Non-ICGN | 43 | 8.1 (1.0-41.4) | 0 (0-6) | 0.0 (0.0-3.2) |
| ICGN | 57 | 6.8 (1.0-41.4) | 0 (0-7) | 0.0 (0.0-7.8) |

*Number of bands >200 kDa and RSA200 were significantly greater in MPGN vs. non-MPGN dogs

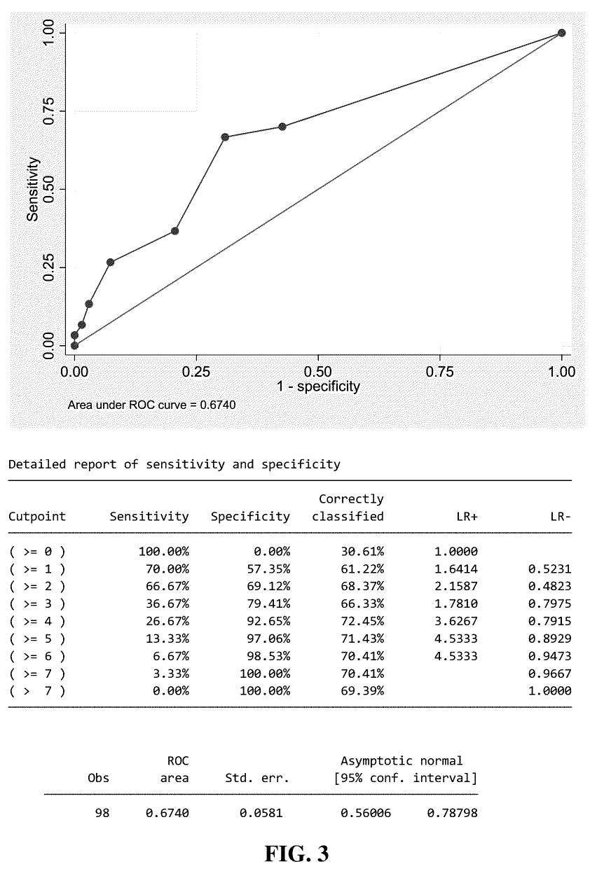

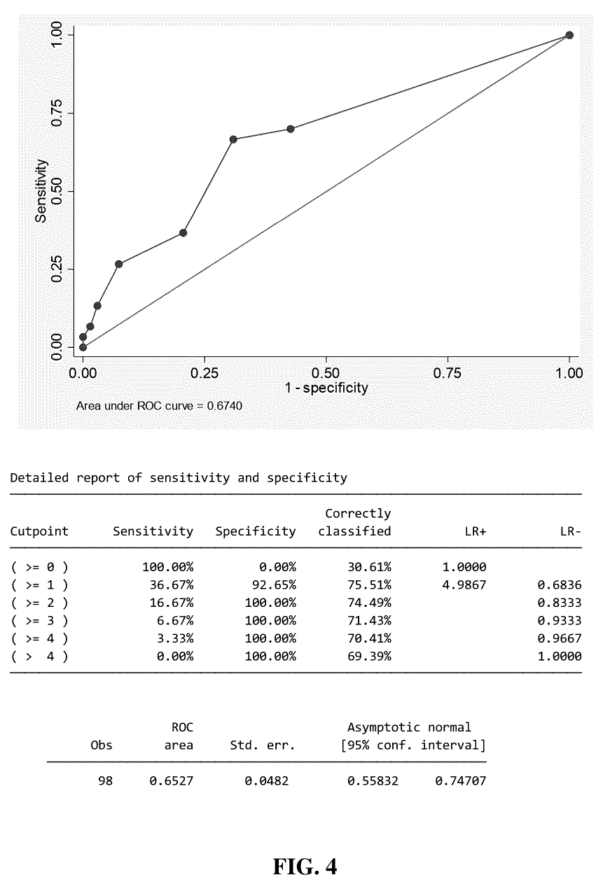

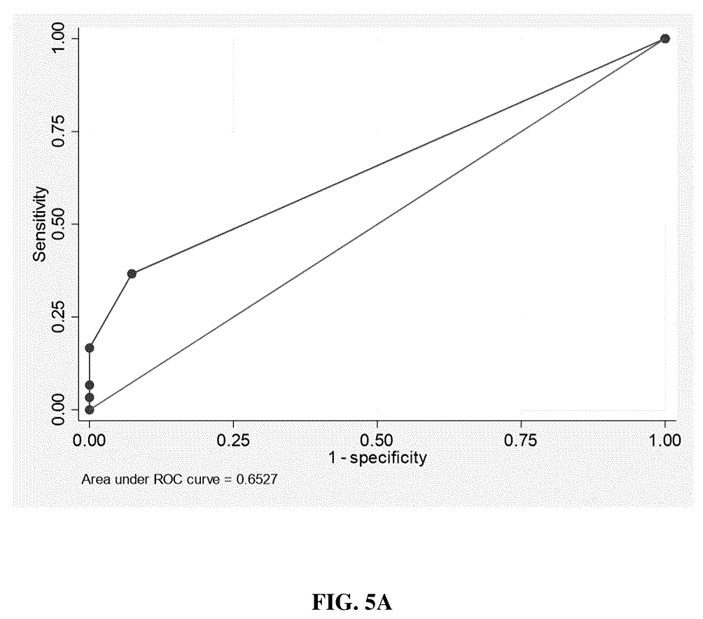

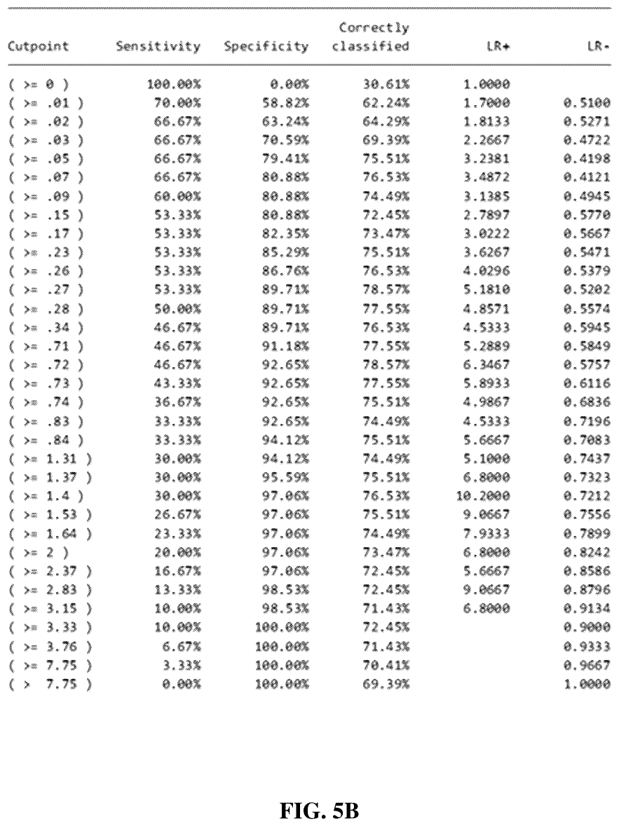

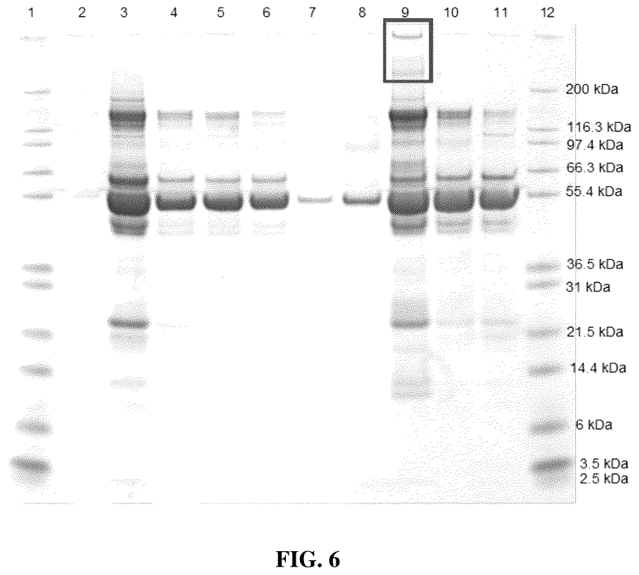

As shown in FIG. 3, the analysis demonstrates that ≥4 protein bands in a sample observed to be greater or equal to 200 kDa via SDS-PAGE is specific for MPGN (93%; CI: 86-99%). FIG. 4 also shows that ≥1 protein band in a sample observed to be greater or equal to 300 kDa via SDS-PAGE is similarly specific for MPGN (93%; CI: 86-99%). Furthermore, FIGS. 5A and 5B demonstrate that a RSA200 value of ≥0.72 in a sample is also specific for MPGN (93%; CI: 86-99%). These results indicate that presence of multiple high molecular weight protein bands detectable by SDS-PAGE and conventional protein staining reagents, particularly larger than 200-300 kDa, in the urine of proteinuric dogs with suspected glomerular disease is highly specific for MPGN, and immunosuppression may be recommended as part of therapy.

UPC did not differ significantly between dogs with ICGN and non-ICGN. Contrary to a previous study (Aresu, L. et al. (2017) Journal of Veterinary Internal Medicine. 31:459-1468) which found that only dogs with ICGN had a UPC >12.5, in the present study, dogs with amyloidosis had a UPC as high as 41.1 (median 10.4) and dogs with FSGS had a UPC as high as 20.6 (median 7.2). Schneider et al (2013) found that only 48.1% of 501 proteinuric dogs biopsied for suspicion of glomerular disease had ICGN. In this study, dogs with ICGN had UPCs ranging from 0.6-42.7 while those with non-ICGN diseases had UPCs ranging from 0.5-40.1. Based on this data, UPC cannot be recommended as a biomarker to distinguish ICGN from non-ICGN glomerular diseases d in dogs.

In contrast, the results presented herein demonstrate that a significant difference in SDS-PAGE banding patterns exists between dogs with MPGN and non-MPGN. Using optimal cut-off values of ≥4 for number of bands above 200 kDa and ≥0.72% for RSA200, each resulted in a 93% specificity for MPGN (sensitivity at these cut-off values was <50%). In view of these results, urine SDS-PAGE can provide sufficient evidence to aid treatment decisions, including starting empiric immunosuppressive therapy, absent a pathologic diagnosis when either ≥4 bands above 200 kDa or an RSA200>0.72% is identified. The present disclosure therefore provides the opportunity to identify and treat patients to improve and prolong quality of life without the need for a comprehensive renal biopsy.

### Example 3

Materials and Methods for Investigating Electrophoretic Urine Protein Banding Patterns in Healthy Cats and Cats with Kidney or Urinary Tract Diseases

### Sample Categorization

Prospective (n=7) and archived (n=79) urine samples from 86 cats were categorized into groups by clinician confirmation and biopsy, with or without bloodwork and urinalysis (UA): normal (n=23), pyelonephritis/UTI (n=11), and CKD (n=37). Archived urine samples from 2008-2023 were obtained from International Renal Veterinary Pathology Service (IVRPS) at Texas A&M University Small Animal Hospital, College Station, TX; the Department of Veterinary Clinical Sciences, Ohio State University Veterinary Medical Center, Columbus, OH; and Colorado State University CVM, Clinical Sciences, Fort Collins, Colorado, USA. Samples were shipped overnight on dry ice to the IVRPS and stored at −80° C. Results from serum chemistry, UAs, and UPCs were documented for samples, if available. All samples were from female and male neutered cats aged 1-19 years.

### Sample Collection and Processing-Healthy Cats

Blood and urine samples from 10 clinically healthy cats were collected between Jul. 18-26, 2023, at the Texas A&M (TAMU) Veterinary Medical Teaching Hospital (VMTH).

Approximately 3 mL of urine was collected via cystocentesis. The urine remained in the syringe used for collection, was capped with a sterile needle, transferred to 15 mL conical tubes (15 mL Centrifuge Tube; VWR, Radnor, PA). A complete UA was performed on whole urine at room temperature (25° C.). Whole urine was used to measure urine specific gravity (USG) by refractometer (Temperature Compensated Hand-Held Refractometer; Reichert Technologies, Depew, NY). A dipstick analysis (Multistix 10 SG Reagent Strips for Urinalysis; Siemens Healthcare Diagnostics, Erlangen, Germany) was also performed on whole urine. Urine was centrifuged at 400×g for 5 minutes to create a urine sediment, where 80% of supernatant from the original volume was removed for the sediment exam on each sample. Approximately 200 μL of supernatant was sent to the Texas A&M Veterinary Medical Diagnostics Laboratory (TVMDL) in College Station, Texas, to obtain a UPC. The remaining supernatant was aliquoted into 2 mL micro tubes tube (Microtube 2 ml, PP; Sarstedt AG & Co. KG, Numbrecht, Germany) were frozen at −80° C. for storage and SDS-PAGE.

During the same visit to the VMTH, approximately 2.5 mL of blood was collected from each cat via the external jugular vein or a superficial vein from the pelvic limb. The blood was aliquoted into an EDTA purple top tube (Monoject Blood Collection Tube, Glycerin Coated Lavender Stopper; Covidien, Dublin, Ireland) and 2 red top tubes (Monoject Blood Collection Tube, No Additive; Covidien). The blood in the red top tubes was centrifuged at 1,000×g for 10 minutes to remove the serum from the blood clot within 30-60 minutes of collection. The serum, urine supernatant, and whole blood to be submitted were refrigerated at 4° C. until delivery to TVMDL.

Confirmatory clinician exams, blood and UA data was available for 13 of the 20 clinically healthy cats. Exclusion criteria for healthy cats included a serum creatinine of <1.6, a UPC<0.2, and other co morbidities (International Renal Interest Society (IRIS)).

Sample Collection and Processing-Cats with Pyelonephritis or Urinary Tract Infections

Confirmatory clinician diagnoses, blood, and UA data was available for 7 of the 14 cats with pyelonephritis, UTI, or both. The known diagnoses included: UTI and CKD (n=1), acute on CKD and UTI (n=1), only UTI (n=2), UTI suspect pyelonephritis (n=1), pyelonephritis and CKD (n=1), and autopsy-confirmed pyelonephritis (n=1). All samples and results were gathered from archived databases. Three cats were excluded due to samples being obtained after treatment.

Sample Collection and Processing Cats with Non-Proteinuric and Borderline Proteinuric CKD

Cats with non-proteinuric CKD were diagnosed with tubulointerstitial damage (n=30) based on clinician diagnosis, bloodwork, and UA results. Confirmatory biopsies (n=4) of tubulointerstitial diseases (obstructive nephropathy (n=1), interstitial nephritis (n=2), and acute tubular necrosis (n=1)) were also included to create a tubulointerstitial (TI) group category (n=34). Additional bloodwork or UA data was not available for 2 of the 34 cats. All samples and results were gathered from archived databases.

Sample Collection and Processing Cats with Proteinuric CKD

Proteinuric CKD cats (n=7) were diagnosed with glomerular damage based on clinician diagnosis, bloodwork, and UA results. An additional 14 cats had confirmatory biopsies with varying glomerular diseases (ICGN (n=10) and non-ICGN (n=4)) were included in the glomerular damage category (n=21). Bloodwork and/or UA data was available for all 21 cats. All samples and results were gathered from archived databases.

### Archive Sample Analysis

A urine dipstick analysis (Multistix 10 SG Reagent Strips for Urinalysis; Siemens Healthcare Diagnostics, Erlangen, Germany) was performed on all archived samples to determine the urine pH and protein concentration and according to the following scale as indicated per the manufacturer's instructions: negative (0 mg/dL), trace 1+(30 mg/dL), 2+(100 mg/dL), 3+(300 mg/dL), and 4+(≥to 2000 mg/dL). Additionally, approximately 200 μL of archived samples from 2022-2023 (normal n=5, CKD n=8) was sent to TVMDL for UPCs.

### Gel Electrophoresis of Urine Samples

All prospective and archived samples underwent denaturing gel electrophoresis, staining, imaging, and computer analysis to determine the number and MW of protein bands in each sample. Nonreducing, denaturing gel electrophoresis was performed on all urine samples using precast 12-well, 4%-12% Bis-Tris gels (Bolt 4-12% Bis-Tris Gel and Invitrogen PowerEase Touch 120 W Power Supply and Mini Gel Tank; Invitrogen Life Technologies, Carlsbad, CA). Urine samples were diluted with ultrapure water based on the USG to a volume of 30 μL, followed by the addition of 10 μL lithium dodecyl sulfate (LDS) sample buffer for a final volume of 40 μL. The urine volume component was calculated to ensure a minimum of 1 μL urine was used for highly concentrated samples. Samples were heated at 70° C. for 10 minutes, and 20 μL of sample or 5 μL of a molecular weight standard (Mark12 Unstained Standard; Invitrogen Life Technologies) was then loaded into separate gel lanes. Gels were run in duplicate (200V for 35 or 52 minutes depending on the number of gel runs (1 or 2) performed at the time) in SDS buffer (NuPAGE LDS Sample Buffer [4×]; Invitrogen Life Technologies, Carlsbad, CA). Following water washes, gels were stained (Imperial Protein Stain; ThermoFisher Scientific, Rockford, IL) for 2 hours and destained overnight with ultrapure water. Gels were photographed (Gel Doc XR+System; Bio-Rad Laboratories Inc., Hercules, CA) and placed in a solution of 2% glycerol/20% ethanol for 20 minutes and dried between cellulose sheets.

### Analysis of Gels

Digital photographs of the gels were analyzed with Image Lab Software 6.1 (Bio-Rad Laboratories Inc., Hercules, CA, USA). Standard lane bands were used to confirm the MW of protein bands seen in sample lanes. For each sample, quantification and subjective assessment of LMW (i.e. low-molecular weight proteins; ≤40 kDa), IMW (i.e. intermediate-molecular weight proteins; 40-70 kDa), and HMW (i.e. high-molecular weight proteins; ≥70 kDa) band intensity was performed using photographed gel image and dried gels to verify band presence.

### Example 4

### Analysis of Gel Electrophoresis of Cat Urine Samples

### Statistical Analysis

The statistical analyses were performed using a commercially available software package (Stata 18, College Station, TX). A Kruskal-Wallis rank sum test followed by post hoc Dunn's pairwise comparison test was performed to determine the significant differences between the LMW, IMW, and HMW bands between each disease category. Descriptive analysis was also performed across LMW, IMW, HWM bands for each disease category.

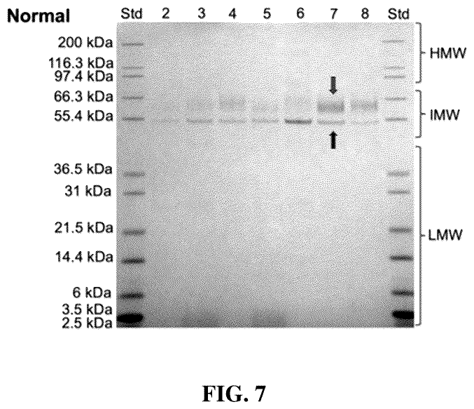

Gel Electrophoresis results from clinically healthy cats consistently showed two bands, a thin band at ˜55 kilodaltons (kDa), which corresponded with albumin protein standard, and a wider hazy band at ˜60 kDa (compatible with cauxin protein) (FIG. 7). The intensity of the presumed cauxin band increased with cat age, especially in male cats. All female and neutered male cats did not have LMW bands except for male one cat. Eleven cats with clinically normal biomarker parameters exhibited a range of 1-4 HMW bands. Six of these cats had no health record data available to confirm clinical health status. Gels from normal cat were significantly different from all diseased categories for LMW and IMW bands but not significantly different from tubulointerstitial damage in HMW bands (Table 4). Descriptive statistical results were recorded (Table 3).

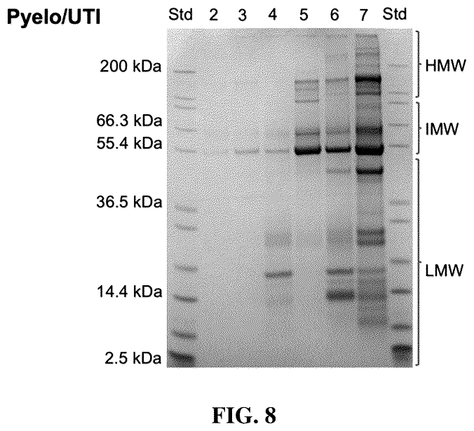

There was a wide range of banding patterns seen in cats with pyelonephritis and/or UTI (Pyelo/UTI; FIG. 8). Bands were significantly different from normal and TI for all LMW, IMW, and HMW and only significantly different from glomerular damage for HMW bands (Table 4).

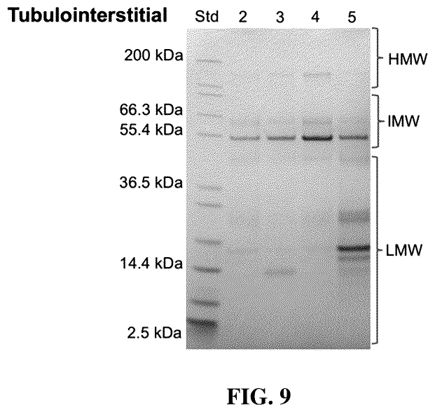

Gels from (TI) cats had a significant increase in LMW and IMW bands but were not significantly different in HMW bands when compared to normal. Band intensity increased as proteinuria increased (FIG. 9). Additionally, a band at ˜150 kDa was consistently seen on all biopsy samples and most non-biopsy samples. Primary tubular damage gels exhibited significant differences in protein bands at IMW and HMW compared to glomerular damage gels and all MW categories to pyelonephritis/UTI (Table 4).

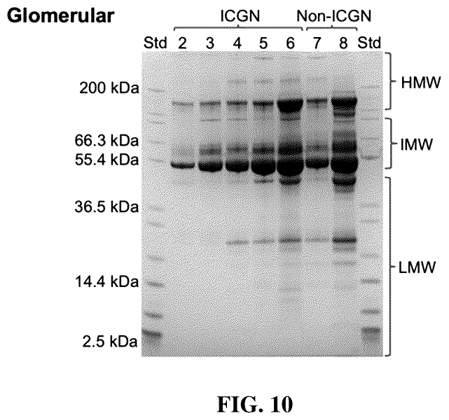

Gels from primary glomerular damage consistently exhibited increased HMW protein bands (FIG. 10). Banding was significantly different from normal and all diseased categories for MW and HMW but not significantly different from pyelonephritis/UTI cats for LMW (Table 4). ICGN and non-ICGN biopsy-confirmed cats showed 1 to 6 HMW bands at >200 kDa. There was no significant difference in protein banding patterns between ICGN and non-ICGN.

TABLE 3
Quantification of LMW, IMW, and HMW protein bands for each sample category.

| | | Normal | | Pyelonephritis/UTI | | Tubulointerstitial | | Glomerular |
|---|---|---|---|---|---|---|---|---|
| | N | Median (Range) | N | Median (Range) | N | Median (Range) | N | Median (Range) |
| LMW | 20 | 0 (0-2) | 11 | 5 (0-11) | 34 | 2 (0-8) | 21 | 5 (1-10) |
| IMW | 20 | 3 (2-4) | 11 | 6 (1-10) | 34 | 4 (2-6) | 21 | 6 (4-9) |
| HMW | 20 | 1 (0-4) | 11 | 5 (0-13) | 34 | 2 (0-7) | 21 | 9 (2-14) |

TABLE 4
Significant difference (*) between disease
categories in LMW, IMW, HMW

| | | Pyelonephritis/ | |
|---|---|---|---|
| | Normal | UTI | Tubulointerstitial |
| LMW | | | |
| Pyelonephritis/ | 0.0000* | | |
| UTI | | | |
| Tubulointerstitial | 0.0001* | 0.0239* | |
| Glomerular | 0.0000* | 0.3621 | 0.0016* |
| IMW | | | |
| Pyelonephritis/ | 0.0002* | | |
| UTI | | | |
| Tubulointerstitial | 0.0185* | 0.0142* | |
| Glomerular | 0.0000* | 0.0992 | 0.0000* |
| HMW | | | |
| Pyelonephritis/ | 0.0006* | | |
| UTI | | | |
| Tubulointerstitial | 0.0536 | 0.0140* | |
| Glomerular | 0.0000* | 0.0368* | 0.0000* |

The normal urine protein banding pattern for cats was similar to dogs with the presence of a compatible albumin band. However, dog urine samples exhibit a consistent band ˜100 kDa consistent with the Tamm-Horsfall protein, that is rarely seen in normal cats. This may mean that cats have a lower concentration of this protein in their urine. The assumed cauxin band in cats is not seen in dogs. Cauxin is a normal pheromone protein found in cat urine and is produced in greater concentrations as a cat ages regardless of sex. However, higher concentrations are expected in intact males. The LMW bands seen in one of the male healthy cats could be an early indicator of tubulointerstitial damage. The eleven cats with HMW bands present may indicate early signs of glomerular damage. The six cats without any historical bloodwork or UA data were deemed clinically healthy cats by clinician assessment. Biopsy confirmation would be needed to confirm if these clinically healthy cats have tubular or glomerular damage present, which were not detected by clinical diagnostic tests. Tubular and glomerular damage protein banding patterns were similar to those seen in dogs. There was no specific banding pattern to distinguish ICGN and non-ICGN in the samples tested; however, low sample size, urine protein degradation in samples stored for long periods of time (10-15 years), and the limited number of confirmatory renal biopsies may have contributed to this result. Even in view of these limitations, however, the results disclosed herein demonstrate that urine SDS-PAGE is a non-invasive method for clinicians to determine the presence of glomerular or tubular damage in cats. The present disclosure therefore provides the opportunity to identify and treat patients to improve and prolong quality of life if a more invasive confirmatory renal biopsy is not possible.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and in the steps or in the sequence of steps of the method described herein without departing from the concept, spirit, and scope of the invention. All such variations and modifications apparent to those skilled in the art are deemed to be within the spirit, scope and concept of the invention as defined by the appended claims.

1. A method of detecting membranoproliferative immune complex-mediated glomerulonephritis (MPGN) in a dog comprising detecting in a sample from said dog the presence of:
a) at least two proteins in the sample having a molecular weight greater than or equal to 200 kDa;
b) at least one protein in the sample having a molecular weight greater than or equal to 300 kDa; or
c) an RSA200 value greater than or equal to 0.7% in the sample wherein said detecting is indicative of MPGN in the dog.

2. The method of claim 1, wherein;
detecting comprises performing electrophoresis on the sample;
the concentration of the protein in the sample is at least 3 ng/μL;
when relative surface area of the protein is quantified, the RSA value is greater than or equal to 0.01%;
the method comprises detecting the presence of at least two proteins in the sample having a mo-lecular weight greater than or equal to 200 kDa; and wherein the at least two proteins are detect-able by SDS-PAGE using a conventional gel staining reagent;
the method comprises detecting the presence of at least one protein in the sample having a molec-ular weight greater than or equal to 300 kDa; and wherein the at least one protein is detectable by SDS-PAGE using a conventional gel staining reagent;
the method comprises detecting the presence of at least three, four, five, six, or seven proteins in the sample having a molecular weight greater than or equal to 200 kDa;
the method comprises detecting the presence of at least two proteins in the sample having a mo-lecular weight greater than or equal to 300 kDa; or
the method comprises detecting an RSA200 value greater than or equal to 0.8%, 0.9%, or 1.0%.

3. The method of claim 2, wherein;
performing electrophoresis comprises sodium dodecyl-sulfate polyacrylamide gel electrophoresis, or
the method further comprises diluting the sample to normalize the sample to urine specific gravity.

4. The method of claim 3, wherein;
detecting further comprises staining the polyacrylamide gel with a gel staining reagent, or
sodium dodecyl-sulfate polyacrylamide gel electrophoresis comprises:
a) electrophoresis on a 4-12% Bis-Tris polyacrylamide gel; or
b) performing electrophoresis at about 200 V.

5-14. (canceled)

15. A method of treating a dog with membranoproliferative immune complex-mediated glomerulonephritis (MPGN) comprising:
a) obtaining a sample from said dog;
b) detecting in the sample the presence of:
i) at least two proteins in the sample having a molecular weight greater than or equal to 200 kDa;
ii) at least one protein in the sample having a molecular weight greater than or equal to 300 kDa; or
iii) an RSA200 value greater than or equal to 0.7% in the sample; and
c) treating the dog based on the presence of (i), (ii), or (iii).

16. The method of claim 15, wherein:
detecting comprises performing electrophoresis on the sample;
treating the dog comprises no treatment;
treating the dog comprises immunosuppressive therapy;
treating the dog does not comprise immunosuppressive therapy;
the sample is a urine sample;
the method further comprises diluting the sample to normalize the sample to urine specific gravity;
the concentration of the protein in the sample is at least 3 ng/μL;
detecting said proteins are detectable by SDS-PAGE using a conventional gel staining reagent; or
when relative surface area of the protein is quantified, the RSA value is greater than or equal to 0.01%.

17. The method of claim 16, wherein performing electrophoresis comprises sodium dodecyl-sulfate polyacrylamide gel electrophoresis.

18. The method of claim 17, wherein detecting further comprises staining the polyacrylamide gel with a gel staining reagent.

19-21. (canceled)

22. The method of claim 16, wherein:
treating the dog comprises administering a angiotensin-a converting enzyme (ACE) inhibitor, a angiotensin receptor blocker, an anticoagulant, an omega-3 fatty acid, a renal disease-specific diet, managing blood pressure, or a combination thereof, or the immunosuppressive therapy comprises mycophenolate, cyclophosphamide, azathioprine, chlorambucil, or cyclosporine.

23-28. (canceled)

29. A method of treating a dog with membranoproliferative immune complex-mediated glomerulonephritis (MPGN) comprising:
a) obtaining a sample from the dog that comprises at least a first protein associated with MPGN;
b) detecting the presence of the at least first protein associated with MPGN in the sample, wherein said protein comprises a molecular weight greater than or equal to 200 kDa; and
c) treating the dog based on the presence of said protein in the sample.

30. The method of claim 29, wherein detecting comprises separating the at least first protein associated with MPGN in the sample based on molecular weight.

31. A method of detecting glomerular or tubular damage in a cat comprising detecting in a sample from said cat the presence of:
a) at least two proteins in the sample having a molecular weight less than or equal to 40 kDa;
b) at least four proteins in the sample having a molecular weight between about 40 kDa and 70 kDa; or
c) at least two proteins in the sample having a molecular weight greater than or equal to 70 kDa;
wherein said detecting is indicative of glomerular or tubular damage in the cat.

32. The method of claim 31, wherein:
detecting comprises performing electrophoresis on the sample;
the sample is a urine sample; or
the method comprises detecting the presence of:
a) at least three proteins in the sample having a molecular weight less than or equal to 40 kDa;
b) at least five proteins in the sample having a molecular weight between about 40 kDa and 70 kDa; or
c) at least three proteins in the sample having a molecular weight greater than or equal to 70 kDa; and
wherein the proteins are detectable by SDS-PAGE using a conventional gel staining reagent.

33. The method of claim 32, wherein performing electrophoresis comprises sodium dodecyl-sulfate polyacrylamide gel electrophoresis.

34-35. (canceled)

36. A method of treating a cat with glomerular or tubular damage comprising:
a) obtaining a sample from said cat;
b) detecting in the sample the presence of:
i) at least two proteins in the sample having a molecular weight less than or equal to 40 kDa;
ii) at least four proteins in the sample having a molecular weight between about 40 kDa and 70 kDa; or
iii) at least two proteins in the sample having a molecular weight greater than or equal to 70 kDa; and
c) treating the cat based on the presence of (i), (ii), (iii) or (iv).

37. The method of claim 36, wherein treating the cat:
comprises no treatment;
comprises immunosuppressive therapy; or
does not comprise immunosuppressive therapy.

38. The method of claim 37, wherein:
treating the cat comprises administering immunosuppressive therapy, angiotensin-converting enzyme (ACE) inhibitors, angiotensin receptor blockers, managing of blood pressure, anticoagulants, managing anemia, managing hydration, managing nausea and/or inappentance, managing phosphorous load, omega-3 fatty acid supplementation, or use of specially formulated diets for renal disease patients, or the immunosuppressive therapy comprises administering glucocorticoids, cyclosporine, chlorambucil, or mycophenolate.

39. (canceled)

40. A method of treating a cat with glomerular or tubular damage comprising:
a) obtaining a sample from the cat that comprises at least a first protein associated with glomerular or tubular damage;
b) detecting the presence of the at least first protein associated with glomerular or tubular damage in the sample, wherein said protein comprises a molecular weight a molecular weight less than or equal to 40 kDa, a molecular weight between about 40 kDa and 70 kDa, or a molecular weight greater than or equal to 70 kDa; and
c) treating the cat based on the presence of said protein in the sample.

41. The method of claim 40, wherein detecting comprises separating the at least first protein associated with glomerular or tubular damage in the sample based on molecular weight.
